(19) United States
(12) Patent Application Publication
(10) Pub. No.: US 2025/0255861 A1
(43) Pub. Date: Aug. 14, 2025

(54) METHODS FOR ADMINISTERING BUPRENORPHINE

(71) Applicant: INDIVIOR UK LIMITED, Hull (GB)

(72) Inventors: Becky BISHOP, North Chesterfield, VA (US); Angela DEVEAUGH-GEISS, North Chesterfield, VA (US); Randal BATENHORST, North Chesterfield, VA (US); Peter C. KYLE, III, North Chesterfield, VA (US); Edward SOTO, North Chesterfield, VA (US)

(21) Appl. No.: 18/931,350

(22) Filed: Oct. 30, 2024

Related U.S. Application Data
(60) Provisional application No. 63/594,785, filed on Oct. 31, 2023.

(51) Int. Cl. A61K 31/4748; A61K 47/22; A61K 47/34

(52) U.S. Cl. CPC A61K 31/4748; A61K 47/22; A61K 47/34

(57) ABSTRACT

The disclosure provides methods for administering buprenorphine in a healthcare setting by an administering practitioner to a patient in need thereof. The methods of the disclosure comprise obtaining buprenorphine from a REMS-certified pharmacy or a REMS-certified healthcare setting. Said administering does not require the administering practitioner to be REMS-certified.

### CROSS REFERENCE TO RELATED APPLICATION

This application claims priority to U.S. Provisional Application No. 63/594,785, filed Oct. 31, 2023, which is incorporated by reference herein.

### TECHNICAL FIELD

The disclosure relates to methods for administering buprenorphine in healthcare settings.

### BACKGROUND

Buprenorphine is a partial agonist at the mu-opioid receptor, and an antagonist at the kappa-opioid receptor. It is approved for treating conditions including acute pain, chronic pain, and opioid use disorder. Buprenorphine may be prescribed to treat moderate to severe opioid use disorder.

Buprenorphine has the molecular formula C₂₉H₄₁NO₄ and is also known as (2S)-2-[17-(Cyclopropylmethyl)-4,5α-epoxy-3-hydroxy-6-methoxy-6α, 14-ethano-14α-morphinan-7α-yl]-3,3-dimethylbutan-2-ol, and is covered by U.S. Pat. Nos. 8,921,387, 8975270, 9272044, 9498432, 9782402, 9827241, 10198218, 10558394, 10592168, 10646484, and 11000520, which are hereby incorporated by reference in their entireties.

Buprenorphine may be administered as an extended-release composition for subcutaneous injection. In some embodiments, the formulation is designed to deliver buprenorphine at a controlled rate over a one-month period. In some embodiments, the composition is SUBLOCADER. In other embodiments, the composition is any commercially available buprenorphine product.

Serious harm or death can result if extended-release buprenorphine is administered by intravenous injection. Extended-release buprenorphine formulations administered by intravenous injection can form a solid mass upon contact with body fluids, and can cause occlusion, local tissue damage, and thrombo-embolic events, including life-threatening pulmonary emboli.

Because of the risk of serious harm or death that could result from intravenous self-administration, access to extended-release buprenorphine formulations is regulated via a restricted program. A Risk Evaluation and Mitigation Strategy (REMS) was required for approval of extended-release buprenorphine formulations. Extended-release buprenorphine was approved with a prior REMS compared to the methods of the disclosure, which the disclosure provided herein improves upon.

Extended-release buprenorphine can also be abused in a manner similar to other opioids. The risk of abuse provides a further need for a REMS.

A REMS is a strategy to manage known or potential risks associated with a drug. A REMS may be required for authorization of certain medicines. In some embodiments, the REMS is required and/or approved by the U.S. Food and Drug Administration (FDA). In some embodiments, a REMS is required to ensure that the benefits of a drug outweigh the risks.

In the US, extended-release buprenorphine has been approved with a REMS requirement. The goal of the REMS is to mitigate the risk of serious harm or death that could result from intravenous self-administration.

A REMS may require healthcare settings, pharmacies, and/or practitioners (e.g., administering practitioners) to be certified within the REMS. The REMS can ensure that healthcare settings and pharmacies are certified, and only dispense buprenorphine to the administering practitioner.

The prior REMS for buprenorphine required the administering practitioner to obtain REMS certification. The requirement for administering practitioners to obtain REMS certification is stringent, and increases the complexity and cost of implementing the REMS, thus increasing the burden on healthcare systems.

A need exists for improved methods for administering buprenorphine in healthcare settings.

The present disclosure satisfies this need by providing robust, simple, and cost-effective methods that achieve a high compliance rate whilst reducing the burden on healthcare systems.

### SUMMARY

The disclosure relates to methods for administering buprenorphine in healthcare settings. The methods of the disclosure are robust, simple, and cost-effective. The methods of the disclosure also achieve a high compliance rate whilst reducing the burden on healthcare systems.

In some embodiments, the disclosure relates to a method of administering buprenorphine in a healthcare setting by an administering practitioner to a patient in need thereof. The method comprises the administering practitioner obtaining buprenorphine directly from a REMS-certified pharmacy and administering the buprenorphine in the healthcare setting by subcutaneous injection to the patient. Prior to the obtaining, the REMS-certified pharmacy received a request for buprenorphine from a prescribing practitioner for a named patient for a scheduled appointment, and the REMS-certified pharmacy coordinated delivery of the requested buprenorphine to the DEA-registered location of the administering practitioner identified in the request. The administering practitioner is DEA-registered and allowed to administer controlled substances following all local, state, and federal regulations, but the administering practitioner is not required to be REMS-certified.

In some embodiments, the disclosure relates to a method of administering buprenorphine in a REMS-certified healthcare setting by an administering practitioner to a patient in need thereof. The method comprises the administering practitioner obtaining buprenorphine directly from a stock within the REMS-certified healthcare setting, and administering the buprenorphine in the REMS-certified healthcare setting by subcutaneous injection to the patient. Said obtaining does not require confirming patient consent, and said administering does not require the administering practitioner to be REMS-certified.

In some embodiments of the disclosure, the buprenorphine is administered to the abdominal region of the patient.

In some embodiments of the disclosure, the buprenorphine is provided in a prefilled syringe.

In some embodiments of the disclosure, the buprenorphine is dissolved in 50:50 poly (DL-lactide-co-glycolide) polymer and N-methyl-2-pyrrolidone.

In some embodiments of the disclosure, the buprenorphine is administered once per month.

In some embodiments of the disclosure, the buprenorphine is administered as two initial monthly doses of 300 mg followed by 100 mg monthly maintenance doses.

In some embodiments of the disclosure, the method further comprises increasing the monthly maintenance dose to 300 mg.

In some embodiments of the disclosure, the buprenorphine is stored locked at 4° C. prior to said obtaining.

In some embodiments of the disclosure, the patient in need thereof is indicated for the treatment of moderate to severe opioid use disorder.

In some embodiments of the disclosure, the buprenorphine is an extended-release formulation.

In some embodiments, the method is practiced according to any of the features disclosed.

### BRIEF DESCRIPTION OF DRAWINGS

The drawings are for illustration purposes only and do not provide any limitation on the disclosure.

FIG. 1 illustrates a method according to the disclosure of obtaining buprenorphine from a REMS-certified pharmacy.

FIG. 2 illustrates a method according to the disclosure of obtaining buprenorphine from a REMS-certified healthcare setting.

FIG. 3 depicts the number of injections dispensed by REMS-certified pharmacies per quarter before and after introduction of the REMS of the disclosure.

FIG. 4 depicts the number of buprenorphine injections distributed by authorized distributors per quarter before and after introduction of the REMS of the disclosure.

### DETAILED DESCRIPTION

The disclosure relates to methods for administering buprenorphine in healthcare settings.

The methods of the disclosure are robust, simple, and cost-effective. The methods of the disclosure achieve a high compliance rate whilst reducing the burden on healthcare systems.

### REMS Certification

To mitigate the risk of serious harm or death that could result from intravenous self-administration of extended-release buprenorphine, extended-release buprenorphine should not be dispensed directly to a patient. Extended-release buprenorphine should only be obtained from a REMS-certified pharmacy or healthcare setting.

In embodiments according to the methods of the disclosure, the pharmacy or healthcare setting from which buprenorphine is obtained is REMS-certified.

A REMS-certified pharmacy or healthcare setting is certified in the REMS program and enrolled with the relevant provider of buprenorphine.

In some embodiments, the relevant provider of buprenorphine is the manufacturer of buprenorphine and/or of the pharmaceutical composition comprising buprenorphine. In some embodiments, the relevant provider is the application holder for FDA approval of the buprenorphine and/or the pharmaceutical composition. In some embodiments, the relevant provider of buprenorphine is the application holder's designee.

In some embodiments, the REMS-certified healthcare setting or pharmacy is enrolled with the relevant provider as part of a restricted pharmacy distribution network that dispenses buprenorphine nationally or regionally.

In some embodiments, the REMS-certified healthcare setting or pharmacy is part of a restricted pharmacy distribution network.

In some embodiments, the REMS-certified healthcare setting or pharmacy is a pharmacy that is enrolled with the relevant provider as party of a restricted pharmacy distribution network that only serves patients within its defined health system and/or network, or typically dispenses to healthcare providers in clinics, treatment centers, long-term care settings, and similar settings, serving a more limited geography than national/regional pharmacies or healthcare settings.

In some embodiments, a healthcare setting's internal policies do not allow controlled substances such as buprenorphine to be delivered from an outside entity, such as a national REMS-certified pharmacy. Therefore, in some embodiments, the disclosure provides a method wherein the healthcare setting is REMS-certified, such that buprenorphine is obtained directly from a stock within the REMS-certified healthcare setting.

In some embodiments, healthcare settings with practices concentrated in mental health and/or addiction treatment settings submit orders to clinics or mental/behavioral health pharmacies. In some embodiments, where the clinic or pharmacy is part of the healthcare setting, the healthcare setting itself is REMS-certified. In some embodiments, wherein the healthcare setting is REMS-certified, a pharmacy and/or clinic that is part of the healthcare setting does not need to be separately REMS-certified.

The REMS-certified healthcare setting or pharmacy follows the REMS to mitigate risks associated with prescribing buprenorphine.

In some embodiments, the requirements of the REMS comprise: buprenorphine is not dispensed directly to the patient; buprenorphine is only administered by an administering practitioner in a healthcare setting; the pharmacy or healthcare setting from which buprenorphine is obtained is REMS-certified; the pharmacy or healthcare setting from which buprenorphine is obtained has established processes and procedures to verify that buprenorphine is provided directly to the location of the administering practitioner for administration by the administering practitioner; the pharmacy or healthcare setting will not distribute, transfer, loan, or sell the buprenorphine; and/or the pharmacy will coordinate delivery of the buprenorphine with the patient's scheduled appointment and deliver to the administering practitioner.

The administering practitioner obtaining buprenorphine from a REMS-certified pharmacy or a stock within the REMS-certified healthcare setting for a specific named patient is not required to be REMS-certified.

In some embodiments, the REMS-certified pharmacy or healthcare setting uses one or more continuously-updated databases to verify applicable registrations prior to dispensing and/or delivering buprenorphine. In some embodiments, said database is the DEA Registration Validation website.

In alternative embodiments, said database is the Substance Abuse and Mental Health Services Administration (SAMHSA) Buprenorphine Pharmacy Look Up website. Suitable databases to verify applicable registrations are known in the art.

A pharmacy or healthcare setting may submit to the application holder and/or the relevant provider for approval of the buprenorphine and/or the pharmaceutical composition in order to obtain REMS certification. In some embodiments, the submission for certification comprises designating an authorized representative, agreeing to train all staff involved in providing buprenorphine to an administering practitioner for administration to a patient in need thereof, and/or ensuring that buprenorphine is never dispensed directly to a patient.

In some embodiments, wherein a healthcare setting obtains REMS-certification, a pharmacy within said healthcare setting may be covered and be REMS-certified, without submitting a separate application to become REMS-certified.

In some embodiments, wherein the authorized representative of a REMS-certified pharmacy or healthcare setting changes, a new REMS-certification enrolment form must be signed and submitted by the new authorized representative.

In some embodiments, the REMS-certification comprises acceptance of audits. In some embodiments, a buprenorphine REMS Audit Plan defines the requirements for accepting audits.

In some embodiments, the audit plan comprises audit classifications, including compliance with REMS requirements.

The methods of the disclosure do not require the administering practitioner to be REMS-certified. In some embodiments, wherein the administering practitioner is not REMS-certified, the administering practitioner is not contracted to comply with the REMS requirements, because the pharmacy or healthcare setting from which buprenorphine is obtained is REMS-certified.

In some embodiments, the administering practitioner is permitted to administer controlled substances following all relevant legislation.

In some embodiments, the administering practitioner is DEA-registered and allowed to administer controlled substances following all local, state, and federal regulations.

The methods of the disclosure wherein the pharmacy or healthcare setting is REMS-certified and wherein the administering practitioner is not required to be REMS-certified provides a simplified method for dispensing and administering buprenorphine, and reduces the burden on healthcare systems.

### Healthcare Setting

The disclosure relates to methods for administering buprenorphine in a healthcare setting.

In some embodiments, the healthcare setting in which buprenorphine is administered is any location in which an administering practitioner may administer controlled substances.

In some embodiments, the healthcare setting is a group practice, Department of Defense (DoD) facility, outpatient clinic, hospital, hospital pharmacy, Veterans Administration (VA) facility, VA pharmacy, opioid treatment program (OTP), closed healthcare system, criminal justice facility, criminal justice facility pharmacy, federally qualified health center (FQHC), FQHC pharmacy, independent practice, institution, institution pharmacy, integrated delivery network (IDN), IDN pharmacy, or other healthcare setting.

In some embodiments, the healthcare setting is a home health visit. In some embodiments, the healthcare setting is a home health visit at which an administering practitioner administers healthcare.

In some embodiments, the healthcare setting is the location of the administering practitioner.

In some embodiments, the healthcare setting in which buprenorphine is administered is distinct from the REMS-certified pharmacy from which buprenorphine is obtained.

In some embodiments, the healthcare setting in which buprenorphine is administered is the REMS-certified pharmacy from which buprenorphine is obtained.

In some embodiments, the healthcare setting in which the buprenorphine is administered is not REMS-certified. In said embodiments, the method of the disclosure comprises obtaining buprenorphine from a REMS-certified pharmacy. In said embodiments, the REMS-certified pharmacy receives a request from a prescribing practitioner for a named patient for a scheduled appointment, and coordinates delivery of the buprenorphine to the location of the administering practitioner identified in the request.

In some embodiments, coordinating delivery by the REMS-certified pharmacy comprises shipping the buprenorphine from the REMS-certified pharmacy to the location of the administering practitioner. In some embodiments, the location of the administering practitioner is DEA-registered.

In some embodiments, the REMS-certified pharmacy coordinating delivery comprises in-store pickup or collection of the buprenorphine from the REMS-certified pharmacy. In some embodiments, the buprenorphine is collected by the administering practitioner or an agent designated by the administering practitioner.

In some embodiments, the healthcare setting is a REMS-certified healthcare setting.

In some embodiments, where the healthcare setting is REMS-certified and holds a stock of buprenorphine, the method comprises obtaining buprenorphine directly from said stock.

In some embodiments, wherein the healthcare setting is REMS-certified, obtaining the buprenorphine does not require confirming patient consent.

### Prescribing Practitioner

In some embodiments, the disclosure relates to a method wherein a request for buprenorphine is received from a prescribing practitioner.

In some embodiments, a prescribing practitioner is allowed by law to prescribe medications for the treatment of opioid use disorder.

In some embodiments, the prescribing practitioner has been approved to prescribe medications for the treatment of opioid use disorder.

In some embodiments, the prescribing practitioner is a physician, a nurse practitioner, a dentist, a nurse midwife, a nurse anesthetist, a clinical nurse specialist, and/or a physician assistant.

In some embodiments, the prescribing practitioner has met the qualifications to prescribe buprenorphine-containing products for opioid-dependence set out by the relevant authority. In some embodiments, the relevant authority is the Drug Enforcement Administration (DEA). In some embodiments, the prescribing practitioner is authorized to dispense controlled substances by the State in which they practice. Relevant authorities in other jurisdictions are known in the art.

In some embodiments, the prescribing practitioner is also the administering practitioner.

In alternative embodiments, the prescribing practitioner is distinct from the administering practitioner. In some embodiments, the prescribing practitioner and the administering practitioner are not the same practitioner.

### Administering Practitioner

The disclosure relates to methods of administering buprenorphine by an administering practitioner. The methods of the disclosure do not require the administering practitioner to be REMS-certified.

In some embodiments, the administering practitioner is a registered practitioner authorized to conduct the administration of controlled substances.

In some embodiments, the administering practitioner is permitted to administer controlled substances following all relevant legislation.

In some embodiments, the administering practitioner is authorized to administer controlled substances under local, state, and federal regulations.

In some embodiments, the administering practitioner is a registered practitioner.

In some embodiments, the administering practitioner is registered with the relevant agency.

In some embodiments the relevant agency is the DEA. In some embodiments, the administering practitioner is DEA-registered.

In some embodiments, the administering practitioner is a physician, a nurse practitioner, a dentist, a clinical nurse specialist, a certified registered nurse anesthetist, a certified nurse midwife, and/or a physical assistant.

In some embodiments, the administering practitioner is the prescribing practitioner.

In alternative embodiments, the administering practitioner is distinct from the prescribing practitioner. In some embodiments, the prescribing practitioner and the administering practitioner are not the same practitioner.

The methods of the disclosure do not require the administering practitioner to be REMS-certified.

In some embodiments, the administering practitioner is not REMS-certified.

The disclosure provides a method of administering buprenorphine in a healthcare setting by an administering practitioner, wherein said administering does not require the administering practitioner to be REMS-certified.

The methods of the disclosure, wherein the administering practitioner is not required to be REMS-certified, reduce the burden on healthcare services for dispensing and administering buprenorphine, whilst retaining a high compliance rate with the REMS.

### Obtaining Buprenorphine

In some embodiments, buprenorphine is obtained from a REMS-certified pharmacy.

In some embodiments, the REMS-certified pharmacy receives a request for buprenorphine from a prescribing practitioner for a named patient for a scheduled appointment.

In some embodiments prior to the obtaining, the REMS-certified pharmacy uses continuously-updated databases to verify applicable registrations prior to dispensing and/or delivering buprenorphine after receiving a request for buprenorphine from a prescribing practitioner for a named patient for a scheduled appointment.

In some embodiments, the REMS-certified pharmacy coordinates delivery of the requested buprenorphine to the location of the administering practitioner identified in the request.

In some embodiments, the location of the administering practitioner is registered with the relevant authority. In some embodiments, the administering practitioner is registered with the relevant authority.

In some embodiments, the relevant authority with which the administering practitioner and/or the administering practitioner's location is registered is the DEA.

In some embodiments, the buprenorphine is delivered to the DEA-registered location of the administering practitioner.

In some embodiments, where a prescribing practitioner and/or an administering practitioner intends only to obtain buprenorphine from a REMS-certified pharmacy for a specific named patient, the prescribing practitioner and/or the administering practitioner is not required to be REMS-certified.

In some embodiments, the healthcare setting is REMS-certified, and the buprenorphine is obtained directly from a stock within the REMS-certified healthcare setting.

In some embodiments, wherein the buprenorphine is obtained directly from a stock within the REMS-certified healthcare setting, obtaining said buprenorphine does not require confirming patient consent.

In some embodiments, wherein the buprenorphine is obtained directly from a stock within the REMS-certified healthcare setting, the administering practitioner is not required to be REMS-certified.

In some embodiments, the REMS-certified pharmacy or healthcare setting may purchase buprenorphine from the manufacturer, wholesaler, or distributor and maintain it in the inventory of the REMS-certified pharmacy or healthcare setting.

In some embodiments, the manufacturer, wholesaler, or distributer validates that the pharmacy or healthcare setting is REMS-certified prior to distributing the buprenorphine.

In some embodiments, the manufacturer, wholesaler, or distributer verifies that the pharmacy or healthcare setting is eligible to receive buprenorphine before distributing the buprenorphine.

### Buprenorphine Formulations

The methods of the disclosure are directed to methods of administering buprenorphine.

In some embodiments, the formulation to be administered is configured for extended release of buprenorphine.

In some embodiments, an extended-release buprenorphine formulation is configured to release buprenorphine in a controlled manner for an extended period following administration.

In some embodiments, the extended-release buprenorphine formulation is configured to release buprenorphine in a controlled manner for a period of at least 1 week, at least 2 weeks, at least 3 weeks, at least 4 weeks, at least 5 weeks, at least 6 weeks, at least 7 weeks, or at least 8 weeks following administration.

In some embodiments, the extended-release buprenorphine formulation is configured for release of buprenorphine in a controlled manner for a period of 1 month following administration.

In some embodiments, the extended-release formulation of buprenorphine comprises at least one polymer and at least one solvent. In some embodiments, the polymer comprises poly(DL-lactide-co-glycolide). In some embodiments, the solvent comprises N-methyl-2-pyrrolidone. In some embodiments, the extended-release formulation comprises 100 mg buprenorphine, 178 mg poly(DL-lactide-co-glycolide), and 278 mg N-methyl-2-pyrrolidone. In some embodiments, the extended-release formulation comprises 300 mg buprenorphine, 533 mg poly(DL-lactide-co-glycolide), and 833 mg N-methyl-2-pyrrolidone. In some embodiments the extended-release formulation of buprenorphine is SUBLOCADE®.

In some embodiments, buprenorphine is administered to the abdominal region of the patient.

In some embodiments, the injection site on the abdomen is between the transpyloric and transtubercular planes, with adequate subcutaneous tissue that is free of skin conditions, such as nodules, lesions, and/or excessive pigment.

In some embodiments, buprenorphine is provided in a prefilled syringe.

In some embodiments, buprenorphine is dissolved in 50:50 poly(DL-lactide-co-glycolide) polymer and N-methyl-2-pyrrolidone.

In some embodiments, the buprenorphine is administered once per month.

In some embodiments, the site for injection is rotated from one injection to another. In some embodiments, a different site is used for each injection relative to the previous injection, the previous two injections, or the previous three injections.

In some embodiments, the buprenorphine is administered as two initial monthly doses of 300 mg, followed by 100 mg monthly maintenance doses.

In some embodiments, the method further comprises increasing the monthly maintenance dose to 300 mg.

In some embodiments, the dose is increased to 300 mg when the patient in need thereof tolerates the 100 mg dose but does not demonstrate satisfactory clinical response.

In some embodiments, the dose is increased to 300 mg when the dosing interval will be two months. In some embodiments, the dosing interval will be two months because the patient is unable to obtain a dose after one month, for example, when the patient is unable to obtain a dose after one month due to extended travel.

In some embodiments, a patient does not demonstrate satisfactory clinical response when said patient self-reports illicit opioid use and/or tests positive in urine or other drug screens for illicit opioid use.

In some embodiments, the buprenorphine is stored locked prior to said obtaining. In some embodiments, the buprenorphine is stored at 4° C. prior to said obtaining.

In some embodiments, the buprenorphine is formulated as SUBLOCADE®.

### Patient in Need Thereof

The methods of the disclosure are directed to administering the buprenorphine to a patient in need thereof.

In some embodiments, the patient in need thereof is indicated for the treatment of opioid use disorder.

In some embodiments, the opioid use disorder is moderate to severe opioid use disorder.

In some embodiments, the patient is indicated for the treatment of moderate to severe opioid use disorder and has initiated treatment with a buprenorphine-containing product, followed by dose adjustment.

In some embodiments, dose adjustment is for a minimum of seven days.

In some embodiments, the patient in need thereof has a contraindication to methadone, has failed methadone treatment, and/or methadone is not available.

In some embodiments, the patient in need thereof is heroin dependent. In some embodiments, the patient in need thereof is a heroin-dependent, hospitalized patient.

In some embodiments, the patient in need thereof has requested discontinuation of buprenorphine. In some embodiments, the patient in need thereof has requested discontinuation from sublingual buprenorphine. some embodiments, the buprenorphine for administration to the patient in need thereof in accordance with the disclosure is an extended-release formulation comprising buprenorphine.

In some embodiments, the patient in need thereof is unstable on transmucosal buprenorphine. In some embodiments, the patient is unstable on transmucosal buprenorphine where they stop taking transmucosal buprenorphine to use illicit drugs. In some embodiments, the patient stops taking transmucosal buprenorphine for more than one day to use illicit drugs.

### Compositions for Use

In some embodiments, the disclosure relates to a composition comprising buprenorphine for use in the treatment of a patient in need thereof by an administering practitioner in a healthcare setting.

In some embodiments, the composition comprising buprenorphine is for use according to the disclosure of the application.

In some embodiments, the composition is for use by (a) obtaining buprenorphine directly from a REMS-certified pharmacy, and (b) administering the buprenorphine in the healthcare setting by subcutaneous injection to the patient, wherein prior to the obtaining (i) the REMS-certified pharmacy received a request for buprenorphine from a prescribing practitioner for a named patient for a scheduled appointment, and (ii) the REMS-certified pharmacy coordinated delivery of the requested buprenorphine to the DEA-registered location of the administering practitioner identified in the request, wherein the administering practitioner is DEA-registered and allowed to administer controlled substances following all local, state and federal regulations, and wherein said administering does not require the administering practitioner to be REMS-certified.

In some embodiments, the composition is for use by (a) obtaining buprenorphine by delivery coordinated directly by a REMS-certified pharmacy to the healthcare setting pursuant to a request by a prescribing practitioner for the buprenorphine for the patient for a scheduled appointment and (b) administering the buprenorphine in the healthcare setting by subcutaneous injection to the patient, wherein (i) the administering practitioner is DEA-registered and allowed to administer controlled substances following all local, state, and federal regulations, and (ii) said administering does not require the administering practitioner to be REMS-certified.

In some embodiments, the disclosure relates to a composition comprising buprenorphine for use in the treatment of a patient in need thereof by an administering practitioner in a REMS-certified healthcare setting. In some embodiments, administering the buprenorphine by the administering practitioner comprises (a) obtaining buprenorphine directly from a stock within the REMS-certified healthcare setting, and (b) administering the buprenorphine in the REMS-certified healthcare setting by subcutaneous injection to the patient, wherein said obtaining does not require confirming patient consent, and wherein said administering does not require the administering practitioner to be REMS-certified.

In some embodiments, the disclosure relates to the use of buprenorphine for the manufacture of a medicament for administration to a patient in need thereof in a healthcare setting by an administering practitioner.

In some embodiments, buprenorphine is for use for the manufacture of a medicament for application according to the disclosure of the application.

In some embodiments, the use of the buprenorphine for the manufacture of a medicament for administration by the administering practitioner comprises (a) obtaining buprenorphine directly from a REMS-certified pharmacy, and (b) administering the buprenorphine in the healthcare setting by subcutaneous injection to the patient, wherein prior to the obtaining (i) the REMS-certified pharmacy received a request for buprenorphine from a prescribing practitioner for a named patient for a scheduled appointment, and (ii) the REMS-certified pharmacy coordinated delivery of the requested buprenorphine to the DEA-registered location of the administering practitioner identified in the request, wherein the administering practitioner is DEA-registered and allowed to administer controlled substances following all local, state and federal regulations, and wherein said administering does not require the administering practitioner to be REMS-certified.

In some embodiments, the disclosure relates to the use of buprenorphine for the manufacture of a medicament for administration to a patient in need thereof in a REMS-certified healthcare setting by an administering practitioner to a patient in need thereof. In some embodiments, administering the buprenorphine by the administering practitioner comprises (a) obtaining buprenorphine directly from a stock within the REMS-certified healthcare setting, and (b) administering the buprenorphine in the REMS-certified healthcare setting by subcutaneous injection to the patient, wherein said obtaining does not require confirming patient consent, and wherein said administering does not require the administering practitioner to be REMS-certified.

In some embodiments, the disclosure relates to methods of administering buprenorphine by an administering practitioner to a patient in need thereof in a healthcare setting, for example for the treatment of moderate to severe opioid use disorder, the method comprising the administering practitioner by (a) obtaining buprenorphine by delivery coordinated directly by a REMS-certified pharmacy to the healthcare setting pursuant to a request by a prescribing practitioner for the buprenorphine for the patient for a scheduled appointment and (b) administering the buprenorphine in the healthcare setting by subcutaneous injection to the patient, wherein (i) the administering practitioner is DEA-registered and allowed to administer controlled substances following all local, state, and federal regulations, and (ii) said administering does not require the administering practitioner to be REMS-certified. In such embodiments, the healthcare setting may or may not be DEA-registered, and can be any location in which an administering practitioner administers a controlled substance, for example a group practice, institution, Department of Defense (DoD) facility, outpatient clinic, hospital, hospital pharmacy, Veterans Administration (VA) facility, VA pharmacy, opioid treatment program (OTP), closed healthcare system, criminal justice facility, criminal justice facility pharmacy, federally qualified health center (FQHC), FQHC pharmacy, independent practice, institution, institution pharmacy, integrated delivery network (IDN), IDN pharmacy, or home health visit. By way of example, the methods may comprise administering buprenorphine to the abdominal region of the patient, optionally with a prefilled syringe. The buprenorphine may optionally be an extended-release formulation, for example configured to release buprenorphine in a controlled manner over a period of at least 1 week, at least 2 weeks, at least 3 weeks, at least 4 weeks, at least 5 weeks, at least 6 weeks, at least 7 weeks, or at least 8 weeks following administration, wherein the buprenorphine is administered once per month. For example, the buprenorphine may be administered as two initial monthly doses, for example 300 mg, followed by monthly maintenance doses, for example 100 mg, which monthly maintenance doses may optionally be increased, for example to 300 mg. In various embodiments, the extended-release formulation comprises 100 mg buprenorphine, 178 mg poly(DL-lactide-co- glycolide), and 278 mg N-methyl-2-pyrrolidone, and in other embodiments the extended-release formulation comprises 300 mg buprenorphine, 533 mg poly(DL-lactide-co-glycolide), and 833 mg N-methyl-2-pyrrolidone.

In some embodiments, the disclosure relates to methods of administering buprenorphine in a REMS-certified healthcare setting by an administering practitioner to a patient in need thereof, for example for the treatment of moderate to severe opioid use disorder, the method comprising the administering practitioner administering the buprenorphine in the REMS-certified healthcare setting by subcutaneous injection to the patient, said buprenorphine having been obtained directly from a stock within the REMS-certified healthcare setting, wherein said buprenorphine was obtained with or without confirming patient consent, and wherein said administering does not require the administering practitioner to be REMS-certified. By way of example, the methods may comprise administering buprenorphine to the abdominal region of the patient, optionally with a prefilled syringe. The buprenorphine may optionally be an extended-release formulation, for example configured to release buprenorphine in a controlled manner over a period of at least 1 week, at least 2 weeks, at least 3 weeks, at least 4 weeks, at least 5 weeks, at least 6 weeks, at least 7 weeks, or at least 8 weeks following administration, wherein the buprenorphine is administered once per month. For example, the buprenorphine may be administered as two initial monthly doses, for example 300 mg, followed by monthly maintenance doses, for example 100 mg, which monthly maintenance doses may optionally be increased, for example to 300 mg. In various embodiments, the extended-release formulation comprises 100 mg buprenorphine, 178 mg poly(DL-lactide-co-glycolide), and 278 mg N-methyl-2-pyrrolidone, and in other embodiments the extended-release formulation comprises 300 mg buprenorphine, 533 mg poly(DL-lactide-co-glycolide), and 833 mg N-methyl-2-pyrrolidone.

In some embodiments, the disclosure relates to methods of administering buprenorphine by an administering practitioner to a patient in need thereof in a DEA-registered healthcare setting, the method comprising the administering practitioner: (a) obtaining buprenorphine by delivery coordinated directly by a REMS-certified pharmacy to the DEA-registered healthcare setting pursuant to a request by a prescribing practitioner for the buprenorphine for the patient for a scheduled appointment; and (b) administering the buprenorphine in the healthcare setting by subcutaneous injection to the abdominal region of the patient for treatment of moderate to severe opioid use disorder; wherein the buprenorphine is dissolved in 50:50 poly(DL-lactide-co-glycolide) polymer and N-methyl-2-pyrrolidone in a prefilled syringe; wherein the administering practitioner is DEA-registered and allowed to administer controlled substances following all local, state, and federal regulations; and wherein said administering does not require the administering practitioner to be REMS-certified. By way of example, the methods may comprise administering buprenorphine to the abdominal region of the patient, optionally with a prefilled syringe. The buprenorphine may optionally be an extended-release formulation, for example configured to release buprenorphine in a controlled manner over a period of at least 1 week, at least 2 weeks, at least 3 weeks, at least 4 weeks, at least 5 weeks, at least 6 weeks, at least 7 weeks, or at least 8 weeks following administration, wherein the buprenorphine is administered once per month. For example, the buprenorphine may be administered as two initial monthly doses, for example 300 mg, followed by monthly maintenance doses, for example 100 mg, which monthly maintenance doses may optionally be increased, for example to 300 mg. In various embodiments, the extended-release formulation comprises 100 mg buprenorphine, 178 mg poly(DL-lactide-co-glycolide), and 278 mg N-methyl-2-pyrrolidone, and in other embodiments the extended-release formulation comprises 300 mg buprenorphine, 533 mg poly(DL-lactide-co-glycolide), and 833 mg N-methyl-2-pyrrolidone.

In some embodiments, the extended release formulation comprises 100 mg to 300 mg buprenorphine, 178 mg poly(DL-lactide-co-glycolide), and 278 mg N-methyl-2-pyrrolidone.

In some embodiments, the extended release formulation comprises (i) about 18 wt % of buprenorphine; (ii) about 32 wt % of a poly(DL-lactide-co-glycolide) copolymer; and (iii) about 50 wt % of N-methyl-2-pyrrolidone.

As used herein, buprenorphine includes both buprenorphine free base and salts thereof.

It is understood that all numbers herein are intended to include both the exact number stated, as well as to be modified by the word “about.” Herein, about 300 mg of buprenorphine is defined as 295 mg to 305 mg, about 100 mg of buprenorphine is defined as 95 mg to 105 mg, about 178 mg poly(DL-lactide-co-glycolide) is defined as 173 mg to 183 mg, about 278 mg N-methyl-2-pyrrolidone is defined as 273 mg to 283 mg, about 18 wt % of buprenorphine is defined as from 17 wt % to 19 wt %, about 32 wt % of a poly(DL-lactide-co-glycolide) copolymer is defined as from 31 wt % to 33 wt %, about 50 wt % of N-methyl-2-pyrrolidone is defined as from 48 wt % to 52 wt %, about 50:50 poly(DL-lactide-co-glycolide) polymer and N-methyl-2-pyrrolidone is defined as about 47:53 to about 53:47, and about 4° C. is defined as 3.8° C. to 4.2° C.

As used herein, the terms “per month,” “monthly,” or the like means administration one time in one month, and at least a second time one month later. “One month” is defined as about 28 days to about 31 days. For example, the first administration may be on January 1, the second on February 1, the third on March 1, etc. The terms “weekly” or “one week” mean 7 days, which may include from 6-8 days.

The term “administering” includes administration of the formulations described herein to the patient, and can include implantation of a slow-release device in the patient. Administering refers to parenteral administration, for example subcutaneously injecting the formulations. Parenteral administration includes, for example, intravenous, intramuscular, intra-arterial, intradermal, intrathecal, subcutaneous, intraperitoneal, intraventricular, or intracranial.

As used herein, the terms “treat,” “treating,” “treatment,” or the like include at least one of (i) preventing a condition from occurring (prophylaxis); (ii) inhibiting a condition and/or arresting its development; and (iii) relieving symptoms associated with a condition.

As used herein, the term “patient” refers to a human.

### EXAMPLES

The disclosure is further illustrated by the following non-limiting examples. The examples serve only for illustration, and are not intended to limit the scope of the disclosure.

### Example 1

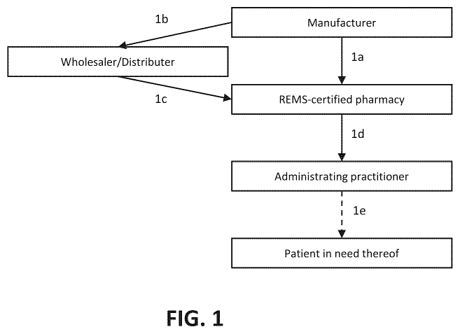

Example 1 provides an exemplary distribution channel for buprenorphine, wherein the buprenorphine is obtained from a REMS-certified pharmacy, as depicted in FIG. 1.

The manufacturer distributes buprenorphine directly to a REMS-certified pharmacy (1a), or the manufacturer distributes buprenorphine to the wholesaler or distributer (1b), and said wholesaler or distributer distributes the buprenorphine to the REMS-certified pharmacy (1c).

When the REMS-certified pharmacy receives a request for buprenorphine from a prescribing practitioner for a named patient for a scheduled appointment, the REMS-certified pharmacy coordinates delivery of the requested buprenorphine to the DEA-registered location of the administering practitioner identified in the request (1d).

The administering practitioner administers the buprenorphine in the healthcare setting to the patient in need thereof (1e). The administering practitioner does not dispense the buprenorphine to the patient.

The solid arrows in FIG. 1 depict physical flow of buprenorphine, and the dotted arrow depicts the flow of service.

### Example 2

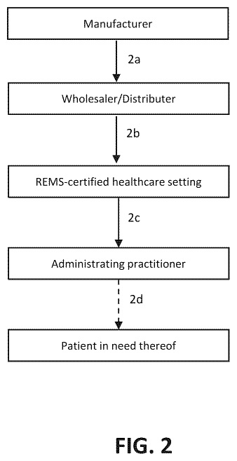

Example 2 provides an exemplary distribution channel for buprenorphine, wherein the buprenorphine is obtained directly from a stock within a REMS-certified healthcare setting, as depicted in FIG. 2.

The manufacturer distributes buprenorphine to the wholesaler or distributor (2a), and said wholesaler or distributer distributes the buprenorphine to the REMS-certified healthcare setting (2b).

The administering practitioner obtains buprenorphine directly from a stock within the REMS-certified healthcare setting (2c) and administers the buprenorphine in the REMS-certified healthcare setting to the patient in need thereof (2d). The administering practitioner does not dispense the buprenorphine to the patient.

The solid arrows in FIG. 2 depict physical flow of buprenorphine, and the dotted arrow depicts the flow of service.

### Example 3

Example 3 provides data demonstrating that the methods of the disclosure, which reduce the burden on healthcare systems, maintain a high compliance rate.

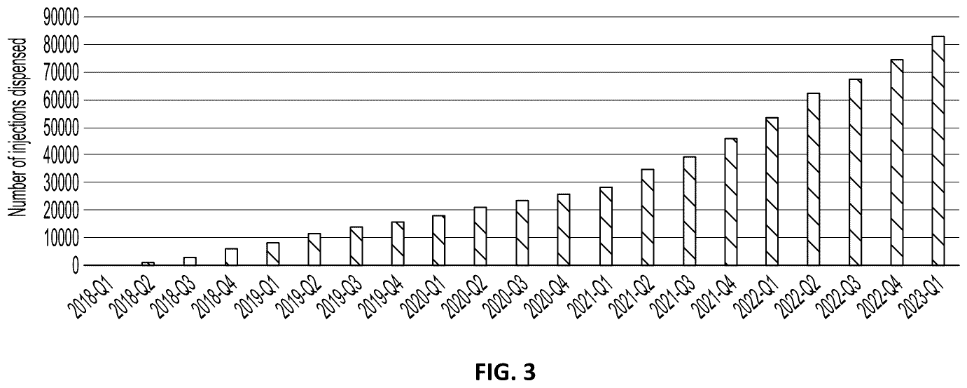

FIG. 3 provides dispensing information from REMS-certified pharmacies. FIG. 3 depicts the number of units of buprenorphine dispensed by REMS-certified pharmacies per quarter before and after introduction of the REMS of the disclosure. Table 1 depicts the number of non-compliance events per quarter before and after introduction of the REMS of the disclosure. A non-compliance event corresponds to buprenorphine being dispensed to a non-healthcare professional. Data for 2023-Q1 in FIG. 3 and Table 1 corresponds to the REMS of the disclosure, and earlier data corresponds to prior REMS.

TABLE 1
Non-compliance associated with REMS-certified
healthcare settings and pharmacies

| | | Percentage of |
|---|---|---|
| Quarter | Number of units | distributions |
| 2018-Q1 | 0 | 0 |
| 2018-Q2 | 1 | 0.086 |
| 2018-Q3 | 0 | — |
| 2018-Q4 | 1 | 0.017 |
| 2019-Q1 | 2 | 0.024 |
| 2019-Q2 | 2 | 0.017 |
| 2019-Q3 | 1 | 0.007 |
| 2019-Q4 | 1 | 0.006 |
| 2020-Q1 | 1 | 0.005 |
| 2020-Q2 | 0 | — |
| 2020-Q3 | 0 | — |
| 2020-Q4 | 1 | 0.004 |
| 2021-Q1 | 0 | — |
| 2021-Q2 | 3 | 0.009 |
| 2021-Q3 | 2 | 0.005 |
| 2021-Q4 | 0 | — |
| 2022-Q1 | 0 | — |
| 2022-Q2 | 0 | — |
| 2022-Q3 | 1 | 0.001 |
| 2022-Q4 | 1 | 0.001 |
| Total for prior REMS | 17 | 0.003 |
| 2023-Q1 | 0 | — |
| Total for REMS of the | 0 | 0 |
| disclosure | | |

The number of injections increased following the introduction of the REMS of the disclosure (FIG. 3), while the number of non-compliance events decreased (Table 1). Seventeen (17) non-compliance events occurred between 2018 and 2022, whereas no non-compliance events occurred with the methods of the disclosure.

FIG. 3 demonstrates that the rate of compliance with the methods of the disclosure is higher than the prior REMS. Despite an increase in the number of injections dispensed, there were fewer non-compliant events. The inventors unexpectedly identified that the REMS could be simplified, as described in the methods of the disclosure, to reduce the burden on healthcare systems, whilst maintaining a high compliance rate.

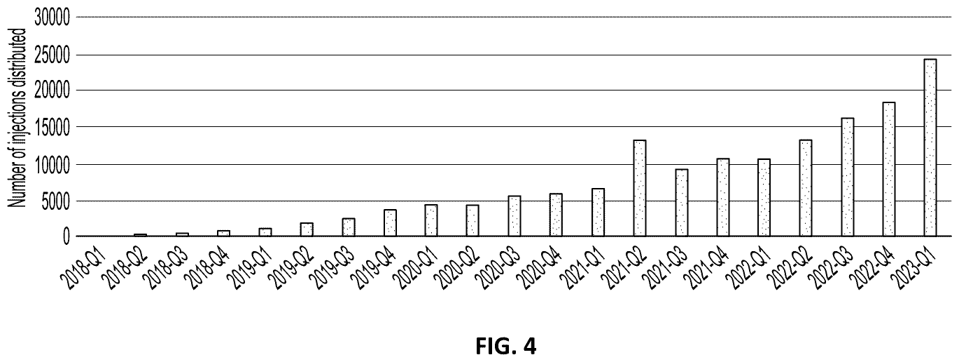

FIG. 4 provides distribution information from authorized distributors to REMS-certified healthcare settings and pharmacies. FIG. 4 depicts the number of buprenorphine injections distributed by authorized distributors per quarter before and after introduction of the REMS of the disclosure. Table 2 depicts non-compliance data for product distributed to a healthcare setting that was not REMS-certified. Table 2 depicts the number of non-compliance events per quarter before and after introduction of the REMS of the disclosure. A non-compliance event corresponds to buprenorphine being distributed to a healthcare setting that was not REMS-certified. Data for 2023-Q1 corresponds to the REMS of the disclosure, and earlier data corresponds to prior REMS.

TABLE 2
Non-compliance events associated with distribution
to non-REMS-certified healthcare settings

| Quarter | Number of units | Percentage of distributions |
|---|---|---|
| 2018-Q1 | | — |
| 2018-Q2 | 2 | 0.697 |
| 2018-Q3 | | — |
| 2018-Q4 | | — |
| 2019-Q1 | | — |
| 2019-Q2 | | — |
| 2019-Q3 | | — |
| 2019-Q4 | 3 | 0.082 |
| 2020-Q1 | | — |
| 2020-Q2 | 10 | 0.229 |
| 2020-Q3 | | — |
| 2020-Q4 | 14 | 0.234 |
| 2021-Q1 | 4 | 0.059 |
| 2021-Q2 | | — |
| 2021-Q3 | | — |
| 2021-Q4 | | — |
| 2022-Q1 | | — |
| 2022-Q2 | | — |
| 2022-Q3 | | — |
| 2022-Q4 | | — |
| 2023-Q1 | 40 | 0.164 |
| Total | 73 | 0.047 |

Between 2018 and 2022, a total of thirty-three (33) non-compliance events occurred. Following introduction of the methods of the disclosure, only a single non-compliant shipment comprising forty (40) units occurred.

The prior REMS provided non-compliant distributions between 0 and 0.697%, and the methods of the disclosure provided non-compliant distributions of 0.164%, demonstrating a favorable compliance rate for the methods of the disclosure.

Therefore, Example 3 demonstrates that the methods of the disclosure provide a simple, robust, and cost-effective method for distributing and administering buprenorphine, that maintains a high compliance rate and represents an improvement over the prior REMS.

Those skilled in the art will recognize many equivalents to the specific embodiments of the disclosure described herein. The scope of the present disclosure is not intended to be limited to the above description.

1. A method of administering buprenorphine by an administering practitioner to a patient in need thereof in a healthcare setting, the method comprising the administering practitioner:
a) obtaining buprenorphine by delivery coordinated directly by a REMS-certified pharmacy to the healthcare setting pursuant to a request by a prescribing practitioner for the buprenorphine for the patient for a scheduled appointment; and
b) administering the buprenorphine in the healthcare setting by subcutaneous injection to the patient,
wherein the administering practitioner is DEA-registered and allowed to administer controlled substances following all local, state, and federal regulations, and
wherein said administering does not require the administering practitioner to be REMS-certified.

2. A method of administering buprenorphine in a REMS-certified healthcare setting by an administering practitioner to a patient in need thereof, the method comprising the administering practitioner administering the buprenorphine in the REMS-certified healthcare setting by subcutaneous injection to the patient, said buprenorphine having been obtained directly from a stock within the REMS-certified healthcare setting,
wherein said buprenorphine was obtained with or without confirming patient consent, and
wherein said administering does not require the administering practitioner to be REMS-certified.

3. The method of claim 1, wherein the buprenorphine is administered to the abdominal region of the patient.

4. The method of claim 1, wherein the buprenorphine is provided in a prefilled syringe.

5. The method of claim 1, wherein the buprenorphine is dissolved in 50:50 poly(DL-lactide-co-glycolide) polymer and N-methyl-2-pyrrolidone.

6. The method of claim 1, wherein the buprenorphine is administered once per month.

7. The method of claim 6, wherein the buprenorphine is administered as two initial monthly doses of 300 mg followed by 100 mg monthly maintenance doses.

8. The method of claim 7, further comprising increasing the monthly maintenance dose to 300 mg.

9. The method of claim 1, wherein the buprenorphine is stored locked at 4° C. prior to said obtaining.

10. The method of claim 1 wherein the patient in need thereof is indicated for the treatment of moderate to severe opioid use disorder.

11. The method of claim 1, wherein the buprenorphine is an extended-release formulation.

12. The method of claim 11, wherein the extended-release buprenorphine formulation is configured to release buprenorphine in a controlled manner for a period of at least 1 week following administration.

13. The method of claim 11, wherein the extended-release formulation comprises 100 mg buprenorphine, 178 mg poly(DL-lactide-co-glycolide), and 278 mg N-methyl-2-pyrrolidone.

14. The method of claim 11, wherein the extended-release formulation comprises 300 mg buprenorphine, 533 mg poly(DL-lactide-co-glycolide), and 833 mg N-methyl-2-pyrrolidone.

15. The method of claim 1, wherein the REMS-certified pharmacy or the REMS-certified healthcare setting uses one or more continuously-updated databases to verify applicable registrations prior to dispensing and/or delivering buprenorphine.

16. The method of claim 1, wherein the healthcare setting is any location in which an administering practitioner administers a controlled substance.

17. The method of claim 16, wherein the healthcare setting is a group practice, institution, Department of Defense (DoD) facility, outpatient clinic, hospital, hospital pharmacy, Veterans Administration (VA) facility, VA pharmacy, opioid treatment program (OTP), closed healthcare system, criminal justice facility, criminal justice facility pharmacy, federally qualified health center (FQHC), FQHC pharmacy, independent practice, institution, institution pharmacy, integrated delivery network (IDN), IDN pharmacy, or home health visit.

18. The method of claim 1, wherein the healthcare setting is DEA-registered.

19. The method of claim 1, wherein the prescribing practitioner and the administering practitioner are not the same practitioner.

20. A method of administering buprenorphine by an administering practitioner to a patient in need thereof in a DEA-registered healthcare setting, the method comprising the administering practitioner:
a) obtaining buprenorphine by delivery coordinated directly by a REMS-certified pharmacy to the DEA-registered healthcare setting pursuant to a request by a prescribing practitioner for the buprenorphine for the patient for a scheduled appointment; and
b) administering the buprenorphine in the healthcare setting by subcutaneous injection to the abdominal region of the patient for treatment of moderate to severe opioid use disorder,
wherein the buprenorphine is dissolved in 50:50 poly(DL-lactide-co-glycolide) polymer and N-methyl-2-pyrrolidone in a prefilled syringe,
wherein the administering practitioner is DEA-registered and allowed to administer controlled substances following all local, state, and federal regulations, and
wherein said administering does not require the administering practitioner to be REMS-certified.
